(19) United States
(12) Patent Application Publication
(10) Pub. No.: US 2022/0020485 A1
(43) Pub. Date: Jan. 20, 2022

(54) MEDICAL DEVICE HAVING FAILSAFE STATE MACHINE

(71) Applicant: Fresenius Vial SAS, Brézins (FR)

(72) Inventor: Damien Barbeyrac, Veurey Voroize (FR)

(21) Appl. No.: 17/419,095

(22) PCT Filed: Dec. 13, 2019
(86) PCT No.: PCT/EP2019/085155

§ 371 Date: Jun. 28, 2021

(30) Foreign Application Priority Data
Mar. 8, 2019 (EP) 19305268.5

(51) Int. Cl. G16H 40/63; G16H 20/17; G16H 40/40; A61M 5/172

(52) U.S. Cl. CPC G16H 40/63; G16H 20/17; A61M 2205/16; A61M 5/172; G16H 40/40

(57) ABSTRACT

A medical device (1) comprises a control device (12) for controlling operation of the medical device (1), the control device (12) comprising a first processing unit (120) for controlling a first function of the medical device (1) and a second processing unit (121) for controlling a second function of the medical device (1). The control device (12) comprises a failsafe state machine (122) configured to monitor a first operational status of the first processing unit (120) and a second operational status of the second processing unit (121) and to control a state of the medical device (1) dependent on the first operational status and the second operational status.

The invention relates to a medical device according to the preamble of claim 1 and to a method for operating a medical device.

A medical device of this kind comprises a control device for controlling operation of the medical device, the control device comprising a first processing unit for controlling a first function of the medical device and a second processing unit for controlling a second function of the medical device.

In medical device of this kind may for example be an infusion device, such as a volumetric (peristaltic) infusion pump or a syringe infusion pump. A medical device of this kind however also may be another device such as a rack serving to mechanically hold and organize infusion devices and serving as a communication link for attached infusion devices. A medical device in addition may be a communication device acting together with other medical devices, such as infusion devices, for example within a healthcare environment, such as a hospital.

A medical device such as an infusion device typically comprises multiple processing units embodied by processors for controlling different functions of the medical device. For example, one processor may serve to control sensor devices and actor devices, such as a pumping mechanism, of the medical device, whereas another, second processor may serve to control software applications for operating the medical device.

During operation of the medical device, herein, it must be ensured that the processors function correctly such that, in particular during an ongoing infusion operation, a medical fluid such as a medication or a nutritional solution is correctly administered to a patient. In case a failure of one or both of the process occurs, appropriate counteractions must be taken such that an incorrect administration of a medical fluid to a patient is strictly avoided.

Typically, a watchdog mechanism is nowadays employed to monitor an operational status of an associated processor. By using such a watchdog mechanism it may not easily be possible to monitor several process concurrently, having the effect that potentially only one processor is monitored at a time. In addition, current solutions potentially are not easily adaptable to software constraints and device needs.

There hence is a desire to provide a medical device which may be equipped with a flexible monitoring function allowing to monitor several processors at the same time and which may be adapted to software constraints and device needs in a flexible manner.

It is an object of the instant invention to provide a medical device and a method for operating a medical device which in an easy and reliable manner allow for a monitoring of several processing units of a control device of the medical device.

This object is achieved by means of a medical device comprising the features of claim 1.

Accordingly, the control device comprises a failsafe state machine configured to monitor a first operational status of the first processing unit and a second operational status of the second processing unit and to control a state of the medical device dependent on the first operational status and the second operational status.

In one embodiment, the first processing unit and the second processing unit are embodied by individual processors. The failsafe state machine herein, in one embodiment, is embodied by a programmable component, such as a CPLD (Complex Programmable Logical Device) or FPGA (Field Programmable Gate Array), which is individual to the first processing unit and the second processing unit.

The control device, hence, comprises separate units, namely a first processing unit for controlling a first function of the medical device and a second processing unit for controlling a second function of the medical device and in addition a failsafe state machine configured to monitor the first processing unit and the second processing unit. By using the failsafe state machine the first processing unit and the second processing unit may be monitored in a concurrent fashion, wherein in case of a failure of one or both of the processing units an appropriate action may be taken in order to modify the state of the medical device, in particular in order to bring the medical device into a safe state to avoid incorrect functioning in particular in the context of an infusion operation.

By implementing the failsafe state machine by a programmable component, such as a CPLD or FPGA, the failsafe state machine may easily be adapted to software constraints and device needs, which makes the operation and updating of the medical device and its operating software flexible.

A CPLD is a programmable logic device for implementing programmable logical functions. A CPLD comprises a non-volatile configuration memory and a large number of gates.

An FPGA is an integrated circuit containing an array of programmable logic blocks. Such logic blocks can be configured to perform complex combinational functions, hence allowing a programming of the FPGA for performing specific functions.

The first processing unit, in one embodiment, may be configured to control operation of at least one of a sensor device for measuring a measurement quantity and an actor device for performing a mechanical function of the medical device. An actor device in particular may be part of a pumping mechanism for administering a medical fluid to a patient, the medical device in this case constituting an infusion device for delivering a medical fluid towards a patient. A sensor device in this respect may for example be a force sensor measuring a measuring quantity indicative of a pressure within an infusion line in the context of an infusion operation. The first processing unit hence controls operation of units such as sensor devices or actor devices having a function for performing a real-time action, for example for delivering a medical fluid in the context of an infusion operation.

The second processing unit, in one embodiment, may be configured to control operation of at least one software application of the medical device. The second processing unit hence serves to execute software to perform specific applications, such as a specific infusion routine or the like in the context of an infusion operation. The second processing unit may also control a human machine interface comprising for example a display device and serving as an input to allow a user to input user commands and an output to output information to a user, the display device for example being constituted as a touch-sensitive display serving as an input and output device.

In one embodiment, one or both processing units comprise a watchdog device for monitoring a state of the associated processing unit. For example, the associated processing unit may be configured to trigger a signal in a periodic fashion, for example every 50 ms or 100 ms. Such a signal may be monitored by the associated watchdog device (also denoted as watchdog timer), the watchdog device outputting a failure indication in case a signal from the associated processing unit is not received in an expected, timely fashion. A failure indication, in this way, may be output for example in case a signal comes too late, in case a signal is not received at all or in case a signal is received too early.

The watchdog device of one of the processing units or the watchdog devices of both processing units may be monitored by the failsafe state machine. In case a signal of the watchdog devices of the processing units is received indicating a failure of one or both of the processing units, the failsafe state machine may be configured to take suitable counteractions to counteract the failure of the corresponding processing unit by modifying a state of the medical device, the counteractions being such that a false operation of the medical device, for example in the context of an infusion operation, is avoided and a potentially harmful administration of a medical fluid towards the patient is prevented.

In one embodiment, the failsafe state machine, for controlling a state of the medical device, may be configured to reset the first processing unit, reset the second processing unit, trigger an alarm, switch off an actor device, switch off a human machine interface, switch off a communication interface, and/or enable a switching off of the medical device.

For example, in case a failure of the first processing unit is detected, the failsafe state machine may cause a reset of the first processing unit. If, in the alternative, a failure of the second processing unit is detected, the failsafe state machine may cause a reset of the second processing unit.

If a failure of the first processing unit or the second processing unit is detected, a corresponding alarm may be triggered, wherein the alarm may be different in case a failure of the first processing unit or a failure of the second processing unit occurs. For example, a failure of the first processing unit (for example serving to control operation of sensor devices and/or actor devices) may cause a standard alarm, involving for example a visual alarm indication and a standard acoustic alarm tone. A failure of the second processing unit (for example serving to control operation of a software application of the medical device) may cause an alarm of a higher priority, for example involving a blinking visual alarm indication as well as a high priority acoustic alarm (for example a beeping indicating an urgency of the alarm).

In particular in case a failure of the second processing unit (for example serving to control operation of a software application of the medical device) occurs, a human machine interface, for example a display device of the human machine interface, may be switched off, in order to avoid a displaying of false information to a user.

If a failure of the first processing unit and/or the second processing unit occurs, an actor device such as a motor of a pumping mechanism may be stopped in order to immediately stop an infusion operation. If a failure of the first processing unit and/or the second processing unit occurs, in addition a user may be allowed to switch of the medical device for example by long pressing (for example longer than 2 seconds) a corresponding button of the medical device.

In one embodiment, the failsafe state machine is configured to provide a status signal to at least one of the first processing unit and the second processing unit to indicate a functional status of the failsafe state machine to the at least one of the first processing unit and the second processing unit. The first processing unit and/or the second processing unit hence are enabled to monitor an operational mode of the failsafe state machine, such that the first processing unit and the second processing unit may detect a failure of the failsafe state machine. In case the first processing unit or the second processing unit receives information about a failure of the failsafe state machine, the corresponding processing unit may trigger a suitable counteraction, such as a reset of the failsafe state machine or, as an ultimate ratio, a stopping of the operation of the medical device in order to ensure a safe operation of the medical device.

In one embodiment, the failsafe state machine comprises a backup power supply allowing an operation of the failsafe state machine even in case a main power supply of the medical device fails. The backup power supply may for example have the shape of a (super-)capacitor or a battery (which is rechargeable or not) for storing electrical energy.

The backup power supply beneficially is separate from the main power supply of the medical device such that the failsafe state machine may be supplied with power from the backup power supply independent from the main power supply of the medical device.

In one embodiment, the first processing unit and/or the second processing unit may be configured to activate or deactivate the failsafe state machine. The operational mode of the failsafe state machine hence may be modified by the first processing unit and/or the second processing unit. This in particular may allow a safe startup of the medical device, in particular a booting of the first processing unit and the second processing unit without erroneous interaction by the failsafe state machine.

This is based on the fact that during startup of the medical device a monitoring of the first processing unit and the second processing unit may lead to false results. Hence, during startup (i.e., when powering up the medical device) the failsafe state machine should be disabled in order to allow the first processing unit and the second processing unit to boot until the operating system of the medical device is operational. Once the operating system is operational, the failsafe state machine may be activated such that, from that point on, the operation of the first processing unit and the second processing unit is suitably monitored. The activation of the failsafe state machine herein may be triggered by one of the processing units (which in this case acts as a supervisor) or another entity of the control device such as an additional processor of the control device.

The object is also achieved by means of a method for operating a medical device, the method comprising: controlling, using a control device, operation of medical device, the control device comprising a first processing unit for controlling a first function of the medical device and a second processing unit for controlling a second function of the medical device, and monitoring, using a failsafe state machine of the control device, a first operational status of the first processing unit and a second operational status of the second processing unit and controlling a state of the medical device dependent on the first operational status of the and the second operational status.

The advantages and advantageous embodiments described above for the medical device equally apply also to the method such that it shall be referred to the above in this respect.

The idea underlying the invention shall subsequently be described in more detail with reference to the embodiments shown in the figures. Herein:

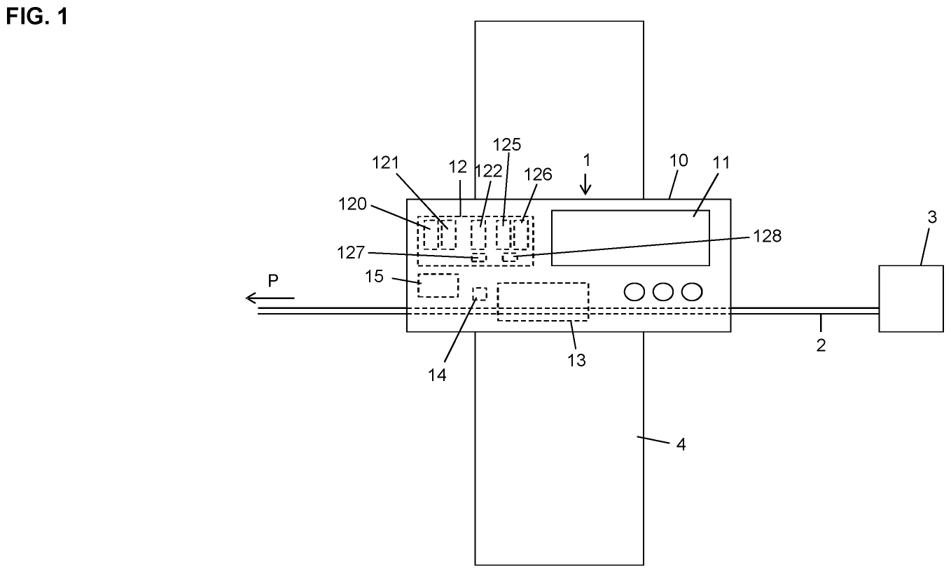

FIG. 1 shows a schematic view of a medical device in the shape of an infusion device for administering a medical fluid to a patient;

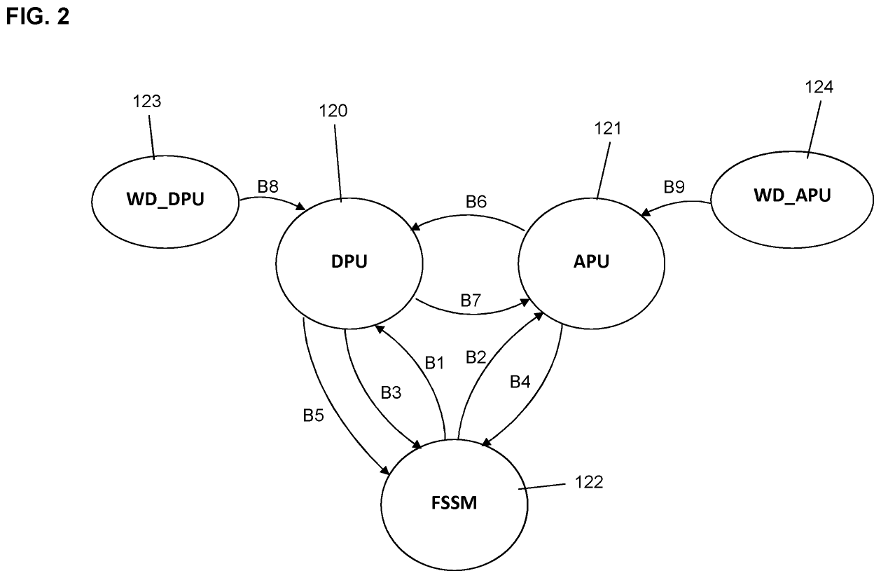

FIG. 2 shows a functional view of a first processing unit in the shape of a delivery processor, a second processing unit in the shape of an application processor and a failsafe state machine of the medical device; and

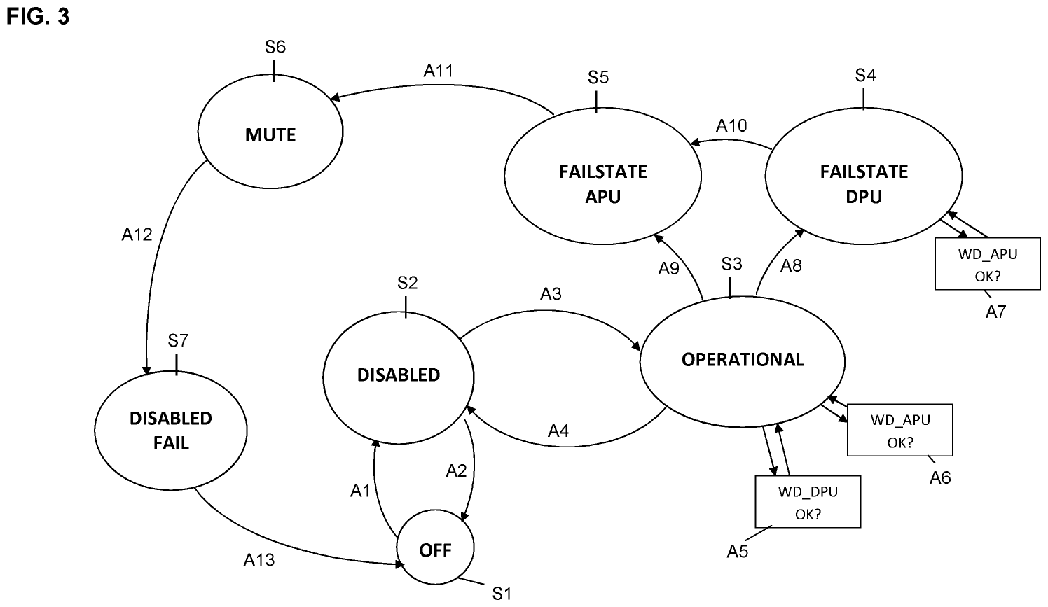

FIG. 3 shows a state diagram of the failsafe state machine.

FIG. 1 shows, in a schematic drawing, a medical device 1 in the shape of an infusion device such as a volumetric (peristaltic) infusion pump.

The medical device 1, in the embodiment of FIG. 1, comprises a housing 10 encompassing an actor device 13 in the shape of a pumping mechanism for acting onto an infusion line of an infusion set 2 connected to a container 3 containing a medical fluid. By means of the pumping mechanism medical fluid may be pumped through the infusion set 2 towards a patient P for delivering medical fluid to the patient P. The medical device 1 herein may be placed on a rack 4 for mechanically holding the medical device 1 potentially together with other medical devices such that the medical devices may for example be organized at the bed side of a patient, for example in an intensive care unit of a hospital.

The medical device 1, in the embodiment of FIG. 1, comprises a human machine interface (in short: HMI) 11 having a display device implemented for example by a touch sensitive display and hence allowing a user to input commands into the medical device 1 as well as displaying information to the user relating to for example an infusion operation conducted by the medical device 1.

The medical device 1 comprises a control device 12 serving to control operation of the medical device 1. The control device 12, in the embodiment of FIG. 1, comprises a first processing unit 120 and a second processing unit 121 in the shape of processors (CPUs). The control device 12 in addition comprises a storage 125 in the shape of a RAM serving as a working memory and a storage 126 in the shape of a ROM serving as a non-volatile memory used to store software, such as an operating system of the medical device 1 and software applications to be executed for operating the medical device 1, for example for administering a medical fluid towards a patient P.

The control device 12, in addition, comprises a failsafe state machine 122 having a backup power supply 127 independent from a main power supply 15 of the medical device 1. The main power supply 15 may for example have the shape of a battery or a supply connection to an external energy network, whereas the backup power supply 127 associated with the failsafe state machine 122 may have the shape of a capacitor or a battery configured to solely supply energy to the failsafe state machine 122 in case of a failure of the main power supply 15.

A communication interface 128 may be implemented by a communication bus or a communication chip for a wireless data communication such as for establishing a Wi-Fi connection or the like to other, external devices.

The processing units 120, 121 of the control device 12 may be dedicated to different functions of the medical device 1.

For example, the first processing unit 120 may be configured to control operation of one or multiple actor devices 13 and/or sensor devices 14, the actor devices 13 for example serving to perform a real-time mechanical action for example in the context of the delivery of a medical fluid through an infusion set 2 and the sensor devices 14 serving to obtain measurement information for example in the context of an infusion operation, a sensor device 14 for example being implemented as a force sensor for sensing a force value on the infusion set 2 indicative of a pressure within the infusion set 2.

The second processing unit 121, in contrast, may be dedicated for executing software applications, for example functional routines in the context of an infusion operation, such as a specific infusion routine relating to a specific drug to be infused to a patient P and defined by a specific infusion protocol involving a particular infusion rate profile and infusion volume, the infusion routine for example being programmed by a user according to input commands input into the medical device 1 by means of the human machine interface 11.

The medical device 1, in the embodiment of FIG. 1, hence comprises multiple processing units 120, 121 serving dedicated functions within the context of operating the medical device 1. The processing units 120, 121, in one embodiment, are implemented by different processor chips and act together, within their specific functionality, to operate the medical device 1. The failsafe state machine 122 herein serves to monitor the processing units 120, 121 in order to detect a potential failure of one or both of the processing units 120, 121, such that the medical device 1 may be placed in a safe state in a reliable fashion in case a failure of one or both of the processing units 120, 121 is detected.

FIG. 2 shows a functional schematic of the processing units 120, 121 in their interaction with the failsafe state machine 122. The failsafe state machine 122, in one embodiment, is implemented by a programmable component such as a CPLD or FPGA and hence is flexibly programmable and adaptable according to device needs and constraints. The failsafe state machine 122 in particular is implemented by an individual component separate to the processing units 120, 121, wherein the individual chips implementing the processing units 120, 121 and the failsafe state machine 122 may for example be placed on a common circuit board (mainboard) of the medical device 1.

In the embodiment of FIG. 2, the first processing unit 120 is denoted as delivery processor (“DPU”) and serves to control operation of actor devices 13 and sensor devices 14. The second processing unit 121 in turn is denoted as application processor (“APU”) and serves to control operation of applications to be executed by the medical device 1 for example in the context of performing infusion operations for administering a medical fluid towards a patient P.

Each processing unit 120, 121, in the embodiment of FIG. 2, comprises a watchdog device 123, 124 serving to monitor an operational state of the associated processing unit 120, 121. Namely, each processing unit 120, 121 is configured to trigger a signal in a periodic fashion, for example every 50 ms or 100 ms, such signal indicating to the associated watchdog device 123, 124 that the processing unit 120, 121 is up and running and functions correctly. The watchdog device 123, 124 (which may be implemented by the same chip as the processing unit 123, 124 or by a separate component) detects whether the signal triggered by the processing unit 120, 121 is received in a timely fashion, and triggers a failure signal in case the signal from the processing unit 120, 121 is received too late, is not received at all or is received too early.

Each watchdog device 123, 124 hence monitors its corresponding processing unit 120, 121 (actions B8, B9 in FIG. 2). In case a watchdog device 123, 124 detects a failure of the associated processing unit 120, 121, the watchdog device 123, 124 may by itself trigger a reset of the corresponding processing using unit 120, 121.

In addition, the processing units 120, 121 may monitor each other to ensure correct functioning of the respective other processing unit 120, 121 (actions B6, B7). In case one processing unit 120, 121 detects a failure of the other processing unit 121, 120, the corresponding processing unit 120, 121 may for example issue an alarm and/or stop operation of actor devices 13 such as a motor of a pumping mechanism in order to stop an ongoing infusion operation.

The failsafe state machine 122 serves to monitor both processing units 120, 121 and hence is configured to monitor multiple processing units 120, 121 concurrently (actions B1, B2). For monitoring the correct functioning of the processing units 120, 121, the failsafe state machine 122 may for example monitor the watchdog devices 123, 124, the failsafe state machine 122 hence detecting a malfunctioning of any one of the processing units 120, 121 according to a failure signal issued by the corresponding watchdog device 123, 124.

In case the failsafe state machine 122 detects a failure of one of the processing units 120, 121, the failsafe state machine 122 may take certain counteractions to prevent a potentially harmful false operation of the medical device 1.

Specifically, if the failsafe state machine 122 detects an error of the first processing unit 120 (DPU), the failsafe state machine 122 may for example inform the other processing unit 121 (APU) of the error of the processing unit 120. The failsafe state machine 122 may in addition trigger a reset of the processing unit 120, may stop an operation of actor devices 13, in particular a motor of a pumping mechanism and hence an ongoing infusion operation. The failsafe state machine 122 may trigger an alarm, such as a standard alarm involving a visual alarm and an acoustic alarm for example by outputting a standard alarm tone. In addition, the failsafe state machine 122 may cause the medical device 1 to be mechanically unlocked from a slot of the rack 4 within which the medical device 1 is received, and a user may be enabled to switch off the medical device 1 for example by a long pressing an off button of the medical device 1.

If, in the alternative, the failsafe state machine 122 detects an error of the processing unit 121 (APU), the failsafe state machine 122 may inform the processing unit 120 (DPU) of the error of the processing unit 121. The failsafe state machine 122 may trigger a reset of the processing unit 121 (APU), and may stop actor devices 13, in particular a motor of a pumping mechanism and hence an ongoing infusion operation. In addition, the failsafe state machine 122 may issue an alarm of a higher priority, indicating that potentially an application failure has occurred which requires immediate attention by skilled personnel, such alarm for example involving a visual alarm (for example a blinking red light) and a high priority acoustic alarm (such as a loud beeping). The failsafe state machine 122 may cause the display of the human machine interface 11 to be switched off, in order to avoid a displaying of any false information to a user. In addition, the failsafe state machine 122 may cause the medical device 1 to be mechanically unlocked from a slot of the rack 4 within which the medical device 1 is received, and a user may be enabled to switch off the medical device 1 for example by long pressing an off button of the medical device 1.

In addition, in one embodiment, the failsafe state machine 122 may itself be monitored by the processing units 120, 121 (actions B3, B4). Specifically, the failsafe state machine 122 may provide a status signal to one or both of the processing units 120, 121 in order to indicate that the failsafe state machine 122 is functioning correctly. If the processing units 120, 121 do not receive such status signal, the processing units 120, 121 hence are enabled to detect that the failsafe state machine 122 does not function correctly. In case one of the processing units 120, 121 detects a failure of the failsafe state machine 122, the corresponding processing unit 120, 121 may inform the other processing unit 121, 120 of the failure of the failsafe state machine 122, may trigger an alarm and potentially may stop actor devices 13, in particular a motor of a pumping mechanism and hence an ongoing infusion operation.

One of the processing units 120, 121 (in the embodiment of FIG. 2 processing unit 120) may in addition be configured to activate or deactivate the failsafe state machine 122 (action B5). In particular, as shall subsequently be described with reference to FIG. 3, during an initial startup of the medical device 1 the failsafe state machine 122 may be disabled until the processing units 120, 121 and an operating system of the medical device 1 are booted, upon which the processing unit 120 activates the failsafe state machine 122 for initiating a monitoring of the processing units 120, 121.

FIG. 3 shows, in a state diagram, states of the failsafe state machine 122 and transitions between the different states of the failsafe state machine 122.

In an initial state S1, when the medical device 1 is switched off, the failsafe state machine 122 is in an OFF state. The medical device 1 in this state is not operational, and the processing units 120, 121 are powered off.

When starting the medical device 1, the failsafe state machine 122 transitions to a DISABLED state S2 (condition A1). In the disabled state the failsafe state machine 122 does not perform any monitoring action and in particular does not monitor the watchdog devices 123, 124 associated with the processing units 120, 121.

The failsafe state machine 122 remains in the disabled state S2 during a startup phase (booting) of the medical device 1. During the startup phase the processing units 120, 121 are powered on and an operating system of the medical device 1 is booted. In addition, software applications are loaded and initiated for execution. Once the processing units 120, 121 are operational, the processing unit 120 (DPU) activates the failsafe state machine 122 such that the failsafe state machine 122 transitions to an OPERATIONAL state S3 (condition A3).

If instead startup does not succeed, for example because power is switched off again, the failsafe state machine transitions back to the OFF state S1 (condition A2).

If the failsafe state machine 122 has transitioned to the operational state S3, but is deactivated again by the processing unit 120, the failsafe state machine 122 transitions back to the disabled state S2 (condition A4).

If the failsafe state machine 122 is in the operational state S3, the failsafe state machine 122 monitors operation of the processing units 120 (APU), 121 (DPU). In particular, the failsafe state machine 122 monitors the watchdog devices 123, 124 for the issuing of a failure signal associated with any of the processing units 120, 121 (conditions A5, A6).

If the failsafe state machine 122 detects a failure of the processing unit 120 (DPU), the failsafe state machine transitions into state S4 (FAILSTATE DPU, condition A8), corresponding to a failstate of the processing unit 120 (DPU). In this state S4 the failsafe state machine 122 may initiate actions defined for a failure of the processing unit 120 (DPU). In particular, as described above, the failsafe state machine 122 may inform the processing unit 121 (APU) of a failure of the processing unit 120 (DPU), may reset the processing unit 120 (DPU), may stop actor devices 13, in particular a motor of a pumping mechanism, may generate a standard alarm, may unlock the medical device 1 from a rack 4, and may authorize a switching off of the medical device 1.

If the failsafe state machine 122, in the operational state S3, detects a failure of the processing unit 121 (APU), the failsafe state machine 122 transitions into state S5 (FAILSTATE APU, condition A9), corresponding to a failstate of the processing unit 121 (APU). In this state S5 the failsafe state machine 122 may take actions associated with and defined for a failure of the processing unit 121 (APU). In particular, the failsafe state machine 122 may inform the other processing unit 120 of a failure of the processing unit 121, may reset the processing unit 121, may stop actor devices 13, in particular a motor of a pumping mechanism, may generate a high priority alarm, may unlock the medical device 1 from a rack 4, may enable a switching off of the medical device 1, and may switch of a display of the human machine interface 11 in order to avoid a displaying of false information to a user.

When in the state S4, the failsafe state machine 122 may in addition monitor a correct functioning of the processing unit 121 (condition A7), such that the failsafe state machine 122 may transition to the state S5 (FAILSTATE APU) in case a failure of also the other processing unit 121 (APU) is detected (condition A10).

If a user mutes the high priority alarm triggered in state S5, the failsafe state machine 122 transitions into a MUTE state S6 (condition A1l).

If a user activates an ON/OFF button of the medical device 1 in order to turn the medical device 1 off, the failsafe state machine 122 transitions into a DISABLED FAIL state S7 (condition A12). Once the medical device 1 is fully switched off (by disconnecting/deactivating the main power supply 15), the failsafe state machine 122 transitions back into its OFF state S1 (condition A13).

Because the failsafe state machine 122 is implemented by a separate component which is flexibly programmable in order to adapt the failsafe state machine 122 to device needs and software constraints, a flexible monitoring of multiple processing units 120, 121 at the same time is enabled.

The embodiments described above are not limiting for the instant invention, but rather the invention may be implemented in an entirely different fashion.

For example, the failsafe state machine may be configured to monitor more than two processing units. The processing units may be dedicated to different or like functions of a medical device. Dependent on the dedicated function and configuration of the processing unit different actions may be triggered by the failsafe state machine in case of a detected failure, wherein the actions are flexibly adaptable according to functional constraints and potentials effects of a malfunctioning of the corresponding processing device.

### LIST OF REFERENCE NUMERALS

1 Medical device
10 Housing
11 Human Machine Interface (display device)
12 Control device
120 Processing unit (delivery processor)
121 Processing unit (application processor)
122 Failsafe State Machine
123, 124 Watchdog device
125 Storage (RAM)
126 Storage (ROM)
127 Backup power supply
128 Communication interface
13 Actor device (pumping mechanism)
14 Sensor device
15 Main power supply (device battery)
2 Infusion set
3 Container
4 Rack
A1-A13 Condition
B1-B9 Action
S1-S7 State
P Patient

1. A medical device, comprising:
a control device for controlling operation of the medical device, the control device comprising a first processing unit for controlling a first function of the medical device and a second processing unit for controlling a second function of the medical device,
wherein the control device comprises a failsafe state machine configured to monitor a first operational status of the first processing unit and a second operational status of the second processing unit and to control a state of the medical device dependent on the first operational status and the second operational status.

2. The medical device according to claim 1, wherein the first processing unit and the second processing unit are embodied by individual processors.

3. The medical device according to claim 1, wherein the failsafe state machine is embodied by a programmable component individual to the first processing unit and the second processing unit.

4. The medical device according to claim 3, wherein the failsafe state machine is embodied by a CPLD or FPGA.

5. The medical device according to claim 1, wherein the first processing unit is configured to control operation of at least one of a sensor device and an actor device for performing a mechanical function.

6. The medical device according to claim 5, wherein the actor device is part of a pumping mechanism for administering a medical fluid to a patient.

7. The medical device according to claim 1, wherein the second processing unit is configured to control operation of at least one software application of the medical device.

8. The medical device according to claim 1, wherein at least one of the first processing unit and the second processing unit comprise a watchdog device for monitoring a state of the associated processing unit, wherein the failsafe state machine is configured, for monitoring the operational status of the associated processing unit, to monitor a signal of the watchdog device.

9. The medical device according to claim 1, wherein the failsafe state machine, for controlling a state of the medical device, is configured to at least one of reset the first processing unit, reset the second processing unit, trigger an alarm, switch off an actor device, switch off a human machine interface, switch off a communication interface, and enable a switching off of the medical device.

10. The medical device according to claim 1, wherein the failsafe state machine is configured to provide a status signal to at least one of the first processing unit and the second processing unit to indicate a functional status of the failsafe state machine to the at least one of the first processing unit and the second processing unit.

11. The medical device according to claim 1, wherein the failsafe state machine comprises a backup power supply separate from a main power supply of the medical device.

12. The medical device according to claim 11, wherein the backup power supply is a capacitor for storing electrical energy.

13. The medical device according to claim 1, wherein at least one of the first processing unit and the second processing unit is configured to activate or deactivate the failsafe state machine.

14. The medical device according to claim 13, wherein during a start-up phase of the medical device the failsafe state machine is disabled, until the failsafe state machine is activated by the at least one of the first processing unit and the second processing unit.

15. A method for operating a medical device, comprising:
controlling, using a control device, operation of the medical device, the control device comprising a first processing unit for controlling a first function of the medical device and a second processing unit for controlling a second function of the medical device, and
monitoring, using a failsafe state machine of the control device, a first operational status of the first processing unit and a second operational status of the second processing unit and controlling a state of the medical device dependent on the first operational status and the second operational status.
